(19) United States
(12) Patent Application Publication
(10) Pub. No.: US 2021/0311007 A1
(43) Pub. Date: Oct. 7, 2021

(54) PORTABLE FUME SENSING DEVICE

(71) Applicant: Telesafe LLC, Cadiz, KY (US)

(72) Inventor: Roland E. GALBREATH, Cadiz, KY (US)

(73) Assignee: Telesafe LLC, Cadiz, KY (US)

(21) Appl. No.: 16/838,051

(22) Filed: Apr. 2, 2020

(51) Int. Cl. G01N 33/00

(52) U.S. Cl. CPC G01N 33/0057

(57) ABSTRACT

A portable fume sensing device. The portable fume sensing device includes a user-proximal end adapted to be held by a user of the portable fume sensing device, a sensing end connected to the user-proximal end by a stem and disposed distally from the user, and a hydrocarbon fume sensor located at the sensing end. A fan is disposed at the sensing end in a user-distal direction from the fume sensor, the fan impelling air towards the fume sensor from a location located in a user-distal direction from the fan. The longitudinal axis of the stem is oriented substantially diagonally with respect to the plane of the fan. An enclosure containing visual indicators, audible indicators, and user operable controls is located at the user-proximal end, the enclosure being coupled to a grip portion that is adapted to be held by a user.

### BACKGROUND

Fire is one of the largest risks to life and property at boating marinas. With numerous boats in close proximity to each other, as well as refueling facilities, a fire starting on one boat has a large likelihood of spreading to others. Awareness and prevention greatly mitigate the risk and occurrence of fire incidents.

The major cause of boat explosions are gasoline fumes. Fuel leaks, aging fuel and vent lines, brittle hoses, and aging fittings can all contribute to the buildup of gasoline or gasoline fumes in the bilge of a boat. Once gasoline fumes are present in the bilge, a mere spark may cause an explosion or a fire. Marine bilge blowers that are inoperable, or that are not allowed to exchange bilge air for the full four minutes as recommended, are another common cause for marine vessel fires. The use of non-marine-rated equipment in boats further increases the risk of catastrophe.

The likelihood of fire due to spark and gasoline vapor is even greater at the fuel dock of a marina. Typically, dockhands receive boats and secure them prior to fueling. The boater is requested to turn off all engines, generators, and close all hatches, windows and doors. The dockhand boards the boat, fuels it, and steps off. These employees are at the greatest risk as they fuel inboard boats and depend on the boat operator to properly execute standard engine starting practices which is to run bilge blowers for a minimum of four minutes prior to starting engines.

A solution that can easily detect the presence of gasoline fumes and lower risk to marina employees is therefore desired.

### SUMMARY

According to at least one exemplary embodiment, a portable fume sensing device is disclosed. The portable fume sensing device can include a user-proximal end adapted to be held by a user of the portable fume sensing device, a sensing end connected to the user-proximal end by a stem and disposed distally from the user, and a hydrocarbon fume sensor located at the sensing end. A fan can be disposed at the sensing end in a user-distal direction from the fume sensor, the fan impelling air towards the fume sensor from a location located in a user-distal direction from the fan. The longitudinal axis of the stem may be oriented substantially diagonally with respect to the plane of the fan. The portable fume sensing device can further include components such as a controller, a power source, and an information panel including one or more of a plurality of visual indicators, an audio indicator, and user-operable controls. Such components may be disposed within an enclosure at the user-proximal end, while the enclosure may be coupled to a grip portion that is adapted to be held by a user.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is a schematic representation of an exemplary embodiment of a portable fume sensing device.

FIG. 2A shows an exemplary embodiment of a portable fume sensing device.

FIG. 2B shows an exemplary embodiment of a portable fume sensing device.

FIG. 3A shows the sensing end of an exemplary embodiment of a portable fume sensing device.

FIG. 3B shows the sensing end of an exemplary embodiment of a portable fume sensing device.

FIG. 4 shows an exemplary embodiment of a portable fume sensing device in use.

FIG. 5 shows an exemplary information panel of a portable fume sensing device.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

According to at least one exemplary embodiment, a portable gasoline fume sensing device is disclosed. The portable gasoline fume sensing device can lower the risk to marina personnel by providing a simple and easily portable apparatus for gasoline fume sensing prior to executing fueling operations or starting boat engines or accessory equipment on board a boat. The portable gasoline fume sensing device can allow the user to detect the presence of gasoline fumes within the bilge of a boat, or another desired location, without necessitating that the user board the boat.

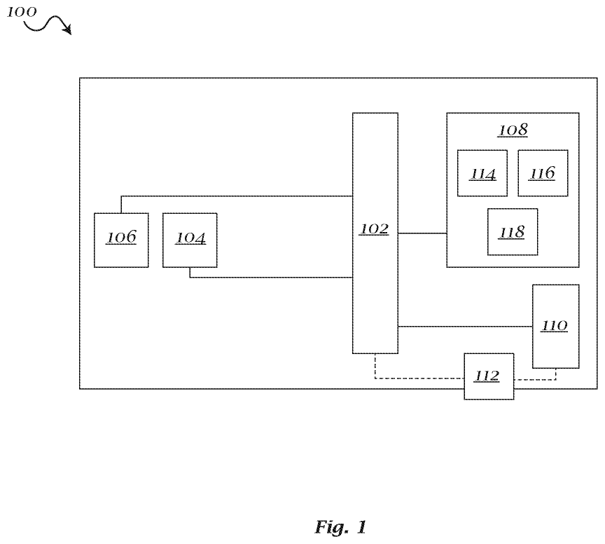

FIG. 1 shows a schematic of an exemplary portable gasoline fume sensing device 100. The sensing device 100 can include a controller 102 which may be communicatively coupled to a fume sensor 104, a fan 106 associated with the fume sensor, and information panel 108. Fume sensor 104 may be any hydrocarbon fume and vapor sensor known in the art that enables sensing device 100 to function as described herein, and may be adapted to sense one or more of gasoline vapor, diesel vapor, propane vapor, compressed natural gas vapor, and so forth. Similarly, fan 106 may be any fan known in the art that enables sensing device 100 to function as described herein, for example a computer or accessory fan. A power source 110, which may be, for example, rechargeable or non-rechargeable batteries, may provide power to all components of sensing device 100. A user-operable switch 112 may be utilized to turn sensing device 100 on and off. The information panel 108 may include one or more visual indicators 114 and one or more audio indicators 116 so as to communicate the status of sensing device 100 to the user. The information panel 108 may further include user operable controls 118. The controller 202 can monitor fume sensor 104, user operable controls 118, and the energy level of power source 110. The controller 202 can also controls the visual indicators 114, audio indicators 116, and fan 106. Controller 202 can determine when an alarm is presented to the user and can set the general system state based on user input, sensor input, and time out conditions.

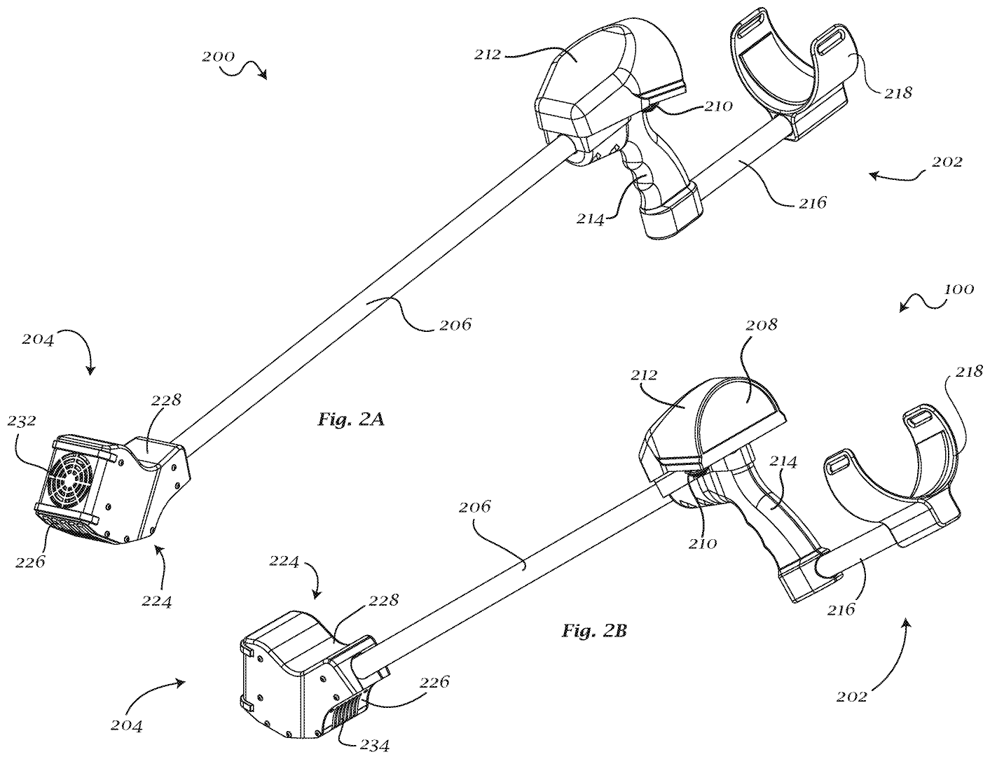

FIGS. 2A-2B show an exemplary embodiment of a portable gasoline fume sensing device 200. The sensing device 200 can include a user-proximal end 202 and a sensing end 204 connected to proximal end 202 by stem 206. Proximal end 202 may be sized and shaped so as to allow sensing device 200 to be comfortably carried by a user and can provide an information panel so as to allow the user to observe the functioning of the sensing device. The various electronic components of sensing device 200, such as controller 102, information panel 208, power source 110, and switch 210 may be disposed within an enclosure 212. The enclosure 212 can further include or be coupled to a grip portion 214 that can allow the user to comfortably hold sensing device 200. Extending rearwardly from grip 214 and towards the user may be a second stem 216 on which an arm rest 218 may be mounted. Arm rest 218 can increase the comfort of carrying sensing device 200 by bracing against the forearm of a user so as to provide a counter force to the weight of sensing end 204.

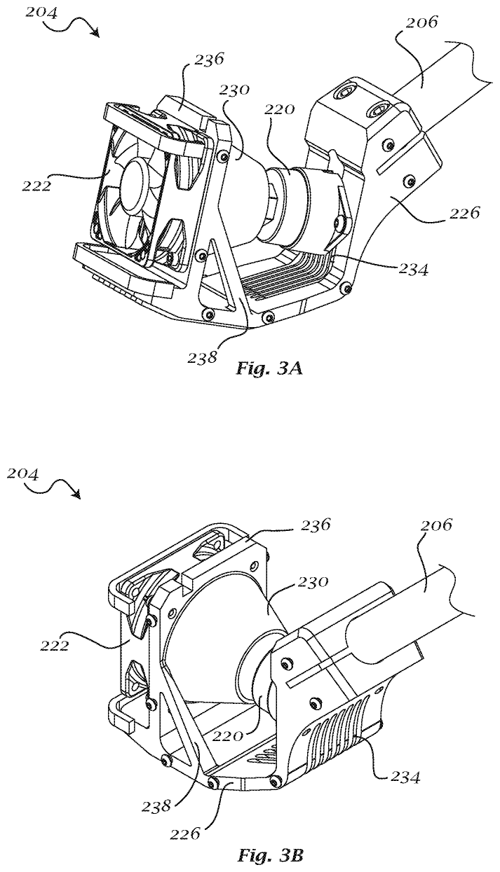

Turning to FIGS. 2A-2B and 3A-3B, located at sensing end 204 may be a fume sensor 220 and fan 222. An housing 224 may be provided to enclose fume sensor 220 and fan 222. The housing 224 may include a mounting portion 226 and a cover portion 228. Mounting portion 226 may be attached to stem 206, while fume sensor 220 and fan 222 may be mounted on mounting portion 226. In some embodiments, fume sensor 220 may have a diameter less than the diameter of fan 222. An air duct 230, which may have a frustoconical shape, may therefore be mounted on mounting portion 226 between fan 222 and fume sensor 220 so as to direct air impelled by fan 222 towards fume sensor 220. Communicative and electrical connections between the various components of proximal end 202 and the various components of sensing end 204 may be disposed within stem 206.

So as to provide airflow through housing 224, an intake grille 232 and an exhaust grille 234 may be defined in housing 224. In one exemplary embodiment, intake grille 232 may be defined in cover portion 228 such that it is disposed forwardly of fan 222, and an exhaust grille 234 may be defined in mounting portion 226 such that it is disposed rearwardly of fan 222. The terms “forwardly” and “rearwardly”, as used herein, should be understood with respect to the airflow through fan 222, as well as with respect to the distance from the user, with “forwardly” being more distal and “rearwardly” being more proximal. A mounting bracket 236 may be provided as part of mounting portion 226, with fan 222 and duct 230 being mounted on bracket 236. Angled supports 238 for bracket 236 may be provided for additional reinforcement.

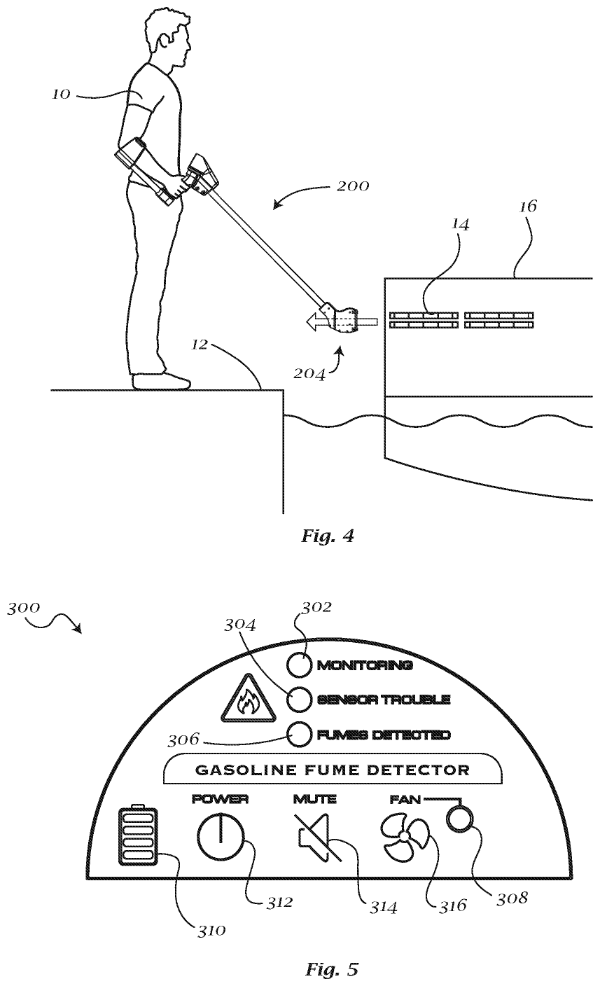

In some exemplary embodiments, housing 224 may be configured such that fan 222 is oriented at an angle with respect to stem 206. That is, as shown in FIG. 4, when stem 206 is disposed approximately diagonally with respect to the horizontal plane, the plane of fan 222 may be substantially vertical. In other words, the longitudinal axis of stem 206 may be oriented substantially diagonally with respect to the plane of fan 222. For example, the angle between the plane of fan 222 and the longitudinal axis of stem 206 may be between 30° and 60°, or between 40° and 50°, or 45°. The fan 222 can therefore draw air in from a location forward of, or user-distal from, sensing end 204. Thus, a user 10 that is standing on a dock, seawall, or other surface 12 external to a boat 16 can comfortably position sensing end 204 proximate a bilge vent 14 of the boat 16 while maintaining sufficient distance from the boat. The user can further maintain a comfortable standing position and a comfortable, relaxed arm position with the arm oriented substantially diagonally to the horizontal plane. Furthermore, while fume sensing device 220 may be successfully used without fan 222 being operational, the fan 222 may be used, for example, to provide active airflow across sensor 220 in situations where existing ambient airflow is not sufficient, or to guarantee airflow across sensor 220 in any situation.

Turning to FIG. 5, an exemplary information panel 300 is shown. Information panel 300 may have a plurality of visual indicators so as to inform the user of the operational status of sensing device 200. In one exemplary embodiment, the visual indicators can include monitoring status 302, sensor malfunction 304, fume detection warning 306, fan status 308, and battery status 310. Monitoring status 302 may, for example, be illuminated when sensing device 200 is operational, and may flash prior to sensing device 200 going into sleep mode. Sensor malfunction 304, may, for example, be illuminated if a fault in the fume sensor 220 is detected. Fume detection warning 306, may, for example, be illuminated if the fume sensor 220 detects the presence of gasoline fumes. Additionally, an audible alert, such as a beep or a series of beeps, may sound upon the detection of the presence of gasoline fumes. Fan status 302 may, for example, be illuminated when fan 222 is operational, and may flash prior to fan 222 going into sleep mode. Battery status 310 may display the charge status of power source 110. Information panel 300 may further include user-operable controls, such as power 312, mute 314, and fan 316. Power 312 may allow the user to enable sensing device 200 or to place it into a sleep mode or an off mode. Mute 314 may allow the user to silence the audible indicators of sensing device 200, while fan 316 may allow the user to enable or disable the operation of fan 222.

The various visual indicators of information panel 300 may be provided by any known illuminating device, for example light emitting diodes. Similarly, the audible indicator may be any known sound-emitting device, such as a sound transducer. The user-operable controls of information panel 300 may be any type of control that enables device 200 to function as described herein, for example membrane switches.

In operation, the fume sensing device may function as follows. A user may turn on the fume sensing device 200 by way of power button 210. As fume sensing device 200 initializes, the visual indicators 302, 304, 306 may cycle illumination for a certain period of time, for example 30 seconds. Following the initialization sequence, fume sensing device 200 may be in an operational status with sensor 220 active and monitoring status indicator 302 illuminated. At this point, the audible indicator 116 may be muted and fan 220 may be off. If desired, the operation of the audible indicator and the fan may be toggled by corresponding buttons 314 and 316, respectively. So as to conserve power source 110, the fan may operate for a certain time period, for example 10 minutes after a button was last pressed, and the fan indicator 308 may flash for a time period, for example 30 seconds, prior to turning off. The user may reset this timer by a short press of fan button 316.

When fume sensor 220 is active, any detection of gasoline fumes by sensor 220 beyond a certain threshold may cause the illumination of fume detection warning 306 and the audible indicator (if operational). If, at any point, the fume sensor 220 fails a self-check procedure by the controller 102, the sensor trouble indicator 304 may illuminate.

Once the user completes the fume detection procedure, fume sensing device 220 may be placed into a sleep mode by a long press of power button 312. Alternatively, the unit may self-place into the sleep mode after a certain time period, for example 15 minutes after the last button press, and the monitoring indicator 302 may flash for a time period, for example 30 seconds, prior to entering the sleep mode. The user may reset this timer by a short press of power button 312.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A portable fume sensing device, comprising:
a user-proximal end adapted to be held by a user of the portable fume sensing device;
a sensing end connected to the user-proximal end by a stem and disposed distally from the user; and
a hydrocarbon fume sensor located at the sensing end.

2. The portable fume sensing device of claim 1, further comprising a fan disposed at the sensing end in a user-distal direction from the fume sensor, the fan impelling air towards the fume sensor from a location located in a user-distal direction from the fan.

3. The portable fume sensing device of claim 2, further comprising:
a housing, the housing having a mounting portion and a cover portion removably coupled to the mounting portion;
wherein the fan and the fume sensor are coupled to the mounting portion.

4. The portable fume sensing device of claim 3, further comprising an intake grille and an exhaust grille defined in the housing.

5. The portable fume sensing device of claim 2, further comprising an air duct disposed between the fan and the fume sensor.

6. The portable fume sensing device of claim 2, wherein the longitudinal axis of the stem is oriented substantially diagonally with respect to the plane of the fan.

7. The portable fume sensing device of claim 1, further comprising an information panel disposed at the user-proximal end, the information panel including one or more of a plurality of visual indicators, an audio indicator, and user-operable controls.

8. The portable fume sensing device of claim 1, further comprising:
a controller;
a power source;
an information panel including one or more of a plurality of visual indicators, an audio indicator, and user-operable controls.

9. The portable fume sensing device of claim 8, wherein the controller, power source, and information panel are housed within an enclosure.

10. The portable fume sensing device of claim 9, wherein the enclosure is disposed at the user-proximal end.

11. The portable fume sensing device of claim 9, further comprising a grip portion coupled to the enclosure, the grip portion adapted to be held by a user.

12. The portable fume sensing device of claim 10, further comprising an arm rest coupled to the grip portion.
